(19) United States
(12) Patent Application Publication
(10) Pub. No.: US 2024/0180932 A1
(43) Pub. Date: Jun. 6, 2024

(54) SUBSTITUTED RESORCYLIC ACID COMPOUNDS IN THE MANAGEMENT OF AGEING AND RELATED DISORDERS

(71) Applicant: SOCIETE DES PRODUITS NESTLE S.A., Vevey (CH)

(72) Inventors: MATTHEW SANDERS, Epalinges (CH); DENIS MARCEL BARRON, Lutry (CH); OLIVIER CICLET, Lonay (CH); MARTINE NARANJO PINTA, Morges (CH); KAMILA MULLER, Epalinges (CH); VINCENZO SORRENTINO, Lausanne (CH); ROBIN WILLOWS, Echandens (CH)

(21) Appl. No.: 18/552,055

(22) PCT Filed: Mar. 25, 2022
(86) PCT No.: PCT/EP2022/058003

§ 371 Date: Sep. 22, 2023

(30) Foreign Application Priority Data
Mar. 25, 2021 (EP) 21165056.9

(51) Int. Cl. A61K 31/00; A61P 43/00

(52) U.S. Cl. CPC A61K 31/658; A61P 43/00

(57) ABSTRACT

The present invention relates to composition comprising at least a compound having general formula (I) for use in (i) increasing resistance to age-related pathologies, (ii) improving a physiological state or disorder related to cell ageing, (iii) improving a physiological state linked to metabolic fatigue in one or more cells, (iv) increasing mitochondrial energy in one or more cells, (v) increasing antioxidant capacity, reducing oxidative stress and/or enhancing mitochondrial function, (vi) improving mobility and/or (vii) improving healthspan and/or lifespan in an individual. The present invention also relates to a compound having general formula I for use as an autophagy inducer.

The present invention relates to composition comprising at least a compound having general formula I for use in (i) increasing resistance to age-related pathologies, ii) improving a physiological state or disorder related to cell ageing (iii) improving a physiological state linked to metabolic fatigue in one or more cells, (iv) increasing mitochondrial energy in one or more cells, (v) increasing antioxidant capacity, reducing oxidative stress and/or enhancing mitochondrial function, (vi) improving mobility and/or (vii) improving healthspan and/or lifespan in an individual. The present invention also relates to a compound having general formula I for use as an autophagy inducer.

### BACKGROUND

AMP-activated protein kinase (AMPK) is an evolutionarily conserved master regulator of energy homeostasis that coordinates metabolic pathways in order to balance nutrient supply with energy demand. AMPK is considered a key drug target to combat the growing epidemic of metabolic disorders such as obesity, type 2 diabetes, cardiovascular disease.

Furthermore, when energy supply is low, organisms sometime respond by slowing ageing and then increasing resistance to a diverse range of age-related pathologies. AMPK has been described as a pro-longevity mediator by modulating multiple longevity pathways. Therefore, AMPK has emerged in the field of healthy ageing as a potential druggable target to prolong health span and prevent age-related decline, potentially playing a protective role in neurodegenerative disease, kidney disease, osteoporosis, cardiovascular disease, metabolic disease, and many forms of cancer, for example.

Currently, there are a range of methods that have been demonstrated to improve healthy ageing including dietary restriction (DR), the reduction of food intake without malnutrition. However, these dietary regimes are very difficult to maintain and follow, so research has focused on targeting the mechanisms underpinning this response. AMPK has been implicated in mediating the benefits of some dietary restriction regimes.

There is a clear unmet need for new natural compounds which can e.g. mimic the beneficial effects of caloric restriction or increase lifespan in an individual that specifically target this enzyme and avoid potential side effects.

### SUMMARY OF THE INVENTION

Numerous studies have demonstrated that a wide range of different natural compounds activate AMPK. However, typically, natural compounds do not activate AMPK by directly binding to the enzyme, but instead indirectly activate AMPK by causing perturbations in mitochondrial respiration. This leads to a decrease in ATP levels of the cell and activation of AMPK through AMP/ADP binding to the nucleotide-binding site of the AMPK y subunit.

In the present invention, we have identified natural compounds from the class of substituted resorcyclic acids that may activate AMPK in cells. Moreover, we showed that such compounds increase median and maximum lifespan, as well as protecting against temperature challenge and improving mobility in aged C elegans.

The present disclosure provides a composition comprising at least a compound having the general formula (I) in an effective amount for use in (i) increasing resistance to age-related pathologies, (ii) improving a physiological state or disorder related to cell ageing, (iii) improving a physiological state linked to metabolic fatigue in one or more cells, (iv) increasing mitochondrial energy in one or more cells, (v) increasing antioxidant capacity, reducing oxidative stress and/or enhancing mitochondrial function, (vi) improving mobility and/or (vii) improving healthspan and/or lifespan in an individual.

The compound having the general formula (I),

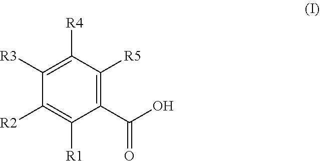

Wherein R1 and R3 are each independently OH; OCH3; O-aliphatic saturated or unsaturated acyl, O-glycoside; acylated O-glycoside; sulfated O-glycoside; or a sulfate.
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.
   or a derivative or analogue thereof.

In some embodiments, a OCH3 group can cyclize with a neighboring OH group to form a methylene dioxy bridge.

In one embodiment, R1 and R3 are each independently OH; OCH3; O-aliphatic saturated or unsaturated acyl, O-glycoside; acylated O-glycoside; sulfated O-glycoside; or a sulfate.
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; an optionally substituted and/or optionally branched C1 to C20 alkyl; an optionally substituted and/or optionally branched, C2 to C20 alkenyl; an optionally substituted and/or optionally branched, C4 to C20 polyalkenyl; an optionally substituted and/or optionally branched C2 to C20 alkynyl, or an optionally substituted and/or optionally branched C4 to C20 polyalkynyl.
   or a derivative or analogue thereof.

In some embodiments, a OCH3 group can cyclize with a neighboring OH group to form a methylene dioxy bridge.

In one embodiment, R1 and R3 are each independently OH; OCH3; O-aliphatic saturated or unsaturated acyl, O-glycoside; acylated O-glycoside; sulfated O-glycoside; or a sulfate.
R2 is H, CH3, OH; O-glycoside; C-glycoside; sulfated O-glycoside; sulfated C-glycoside; a chlorine; a CHO (aldehyde); a sulfate; an optionally substituted and/or optionally branched, C2 to C15 alkenyl; or an optionally substituted and/or optionally branched, C4 to C15 polyalkenyl.
R4 is H; CH3, OH; OCH3; a chlorine, or a 3,3-dimethylallyl chain (1h).
R5 is H; an optionally substituted and/or optionally branched C1 to C20 alkyl; an optionally substituted and/or optionally branched, C2 to C20 alkenyl; or an optionally substituted and/or optionally branched, C4 to C20 polyalkenyl,
   or a derivative or analogue thereof.

In some embodiments, a OCH3 group can cyclize with a neighboring OH group to form a methylene dioxy bridge.

In one embodiment, R1 and R3 are each independently OH; OCH3; O-aliphatic saturated or unsaturated acyl, O-glycoside; acylated O-glycoside; sulfated O-glycoside; or a sulfate.
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C5 isoprenoid chain among the following representatives: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain among the following representatives: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain among the following representatives: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C20 isoprenoid chain among the following representatives: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain among the following representatives: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain among the following representatives: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain among the following representatives: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a); or a derivative or analogue thereof.

In some embodiments, a OCH3 group can cyclize with a neighboring OH group to form a methylene dioxy bridge.

In one embodiment, R1 and R3 are each independently OH; OCH3; O-aliphatic saturated or unsaturated acyl, O-glycoside; acylated O-glycoside; sulfated O-glycoside; or a sulfate.
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C5 isoprenoid chain among the following representatives: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain among the following representatives: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain among the following representatives: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain among the following representatives: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain among the following representatives: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain among the following representatives: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain among the following representatives: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a); or a derivative or analogue thereof.

In some embodiments, a OCH3 group can cyclize with a neighboring OH group to form a methylene dioxy bridge.

In one embodiment, R1 and R3 are each independently OH; OCH3; O-aliphatic saturated or unsaturated acyl, O-glycoside; acylated O-glycoside; sulfated O-glycoside; or a sulfate.
R2 is CH3, OH; O-glycoside; C-glycoside; sulfated O-glycoside; sulfated C-glycoside; a chlorine; a CHO (aldehyde); a sulfate; a C5 isoprenoid chain among the following representatives: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain among the following representatives: 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain among the following representatives: (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); or a (substituted) polyalkenyl chain exclusively represented by: 8-(3,4-dihydroxyphenyl)-4,7-octadien-1-yl (18a);
R4 is H; CH3, OH; OCH3; a chlorine, or a 3,3-dimethylallyl chain (1h);
R5 is H; a C5 isoprenoid chain among the following representatives: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain among the following representatives: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain among the following representatives: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain among the following representatives: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain among the following representatives: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy) pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain among the following representatives: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain among the following representatives: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a), or a derivative or analogue thereof.

In some embodiments, a OCH3 group can cyclize with a neighboring OH group to form a methylene dioxy bridge.

In one embodiment, R1 and R3 are each independently OH; OCH3; O-glycoside; or a sulfate;
R2 is H; 3-methyl-2-buten-1-yl (3,3-dimethylallyl) (1h); or (2E)-3,7-dimethyl-2,6-octadien-1-yl (geranyl) (2h);
R4 is H.
R5 is pentyl; benzyl (6a); 2-phenethyl (6b); or phenylethenyl (14a),
or a derivative or analogue thereof.

In one embodiment, R1=OH; O-glycoside; or a sulfate;
R2 is H; [3-methyl-3-(4-methyl-3-penten-1-yl)-2-oxiranyl]methyl (geranyl 2,3-epoxide) (2e); 3-methyl-6-(1-methylethenyl)-2-cyclohexen-1-yl (2g); (2E)-3,7-dimethyl-2,6-octadien-1-yl (geranyl) (2h); (2Z)-3,7-dimethyl-2,6-octadien-1-yl (neryl) (2i); or 5-methyl-2-(1-methylethyl)cyclohexyl (2o);
R3 is OH; OCH3; O-glycoside; or a sulfate;
R4 is H;
R5 is CH3; n-propyl, n-butyl, or pentyl,
or a derivative or analogue thereof.

In one embodiment, said compound is CBGA (R1=OH; R2 is (2E)-3,7-dimethyl-2,6-octadien-1-yl (geranyl) (2h); R3 is OH; R4 is H and R5 is pentyl).

In one preferred embodiment, said compound is compound 1, CAS number 1244-58-2 Cannabidiolic acid.

Other names:
Benzoic acid, 2,4-dihydroxy-3-[(1R,6R)-3-methyl-6-(1-methylethenyl)-2-cyclohexen-1-yl]-6-pentyl-
Benzoic acid, 2,4-dihydroxy-3-[3-methyl-6-(1-methylethenyl)-2-cyclohexen-1-yl]-6-pentyl-, (1R-trans)-
β-Resorcylic acid, 3-p-mentha-1,8-dien-3-yl-6-pentyl-
2,4-Dihydroxy-3-[(1R,6R)-3-methyl-6-(1-methylethenyl)-2-cyclohexen-1-yl]-6-pentylbenzoic acid
CBDA
Cannabidiol acid
Cannabidiolcarboxylic acid
Cannabinoid CBDA

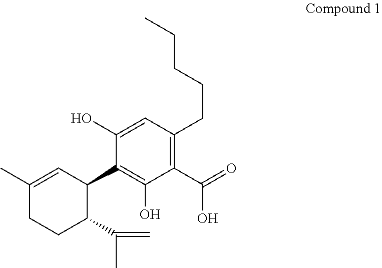 [SMILES: C=C(C)[C@@H]1CCC(C)=C[C@H]1c1c(O)cc(CCCCC)c(C(=O)O)c1O]

An advantage of one or more embodiments provided by the present disclosure is to improve the condition of individuals, animals, humans, ageing animals, or ageing humans.

Still another advantage of one or more embodiments provided by the present disclosure is to reduce or prevent the risk of morbidity or mortality due to excessive catabolism.

An additional advantage of one or more embodiments provided by the present disclosure is to provide beneficial effects of caloric/dietary restriction.

An additional advantage of one or more embodiments provided by the present disclosure is to protect an ageing individual from muscle dysfunction, for example sarcopenia, frailty, inclusion body myositis, myopathy/myolysis induced by drugs such as corticosteroids or statins, muscle wasting induced by immobilization or hospitalization.

An additional advantage of one or more embodiments provided by the present disclosure is to protect an ageing individual from muscle weakness.

Still another advantage of one or more embodiments provided by the present disclosure is to increase the survivability of a critically ill patient or an ageing individual.

An additional advantage of one or more embodiments provided by the present disclosure is to accelerate the regain of mobility, or shorten the time of immobility, after discharge from the intensive care unit.

Additional features and advantages are described in, and will be apparent from, the following Detailed Description and the Figures.

### BRIEF DESCRIPTION OF FIGURES

FIG. 1. Compound 1 improves the thermotolerance of C. elegans, after a 37 C heat-shock. Day 1 adults were treated with 4 different concentrations of Compound 1 for 48 hrs at 20 C. Control is the no-drug-treated population. All compound solutions were made in NGM with 0.2% v/v DMSO, and the bacterial food source (20 mg/mL E. coli OP50). After 48 hrs of Compound 1 treatment, animals were exposed to 37 C for 4 hours and allowed to recover for 15 mins following which death scores were determined using the microfluidic Infinity System. One-way ANOVA test was used to compare the response from different doses. Multiple comparison test was conducted for pairwise comparison to identify the dosages which provide thermotolerance benefit.

FIG. 2. 10 μM Compound 1 extends the mean and maximum lifespan of C. elegans. 60-70 synchronized day 1 adult animals were loaded in each Infinity chip and treated with 0.5 μM or 10 μM of Compound 1, throughout life, and with fresh doses of OP50 bacteria (20 mg/ml) at 20 C, each day. Note that the control is the no drug treated population (0.2% v/v DMSO liquid NGM). All compound solutions were made in sterile, liquid NGM, with 0.2% v/v DMSO co-solvent due to the fact that Compound 1 is hydrophobic with low solubility in water-based solutions. Each day, at the same time, death scores were determined and three independent trials were conducted, with two technical replicates per trial.

FIG. 3. Compound 1 does not extend the lifespan of C. elegans lacking the AMPKα2 subunit (aak-2). 60-70 synchronized day 1 adult aak-2 animals were loaded in each Infinity chip and treated with 0.5 μM or 10 μM of Compound 1, throughout life, and with fresh doses of OP50 bacteria (20 mg/ml) at 20 C, each day. Note that the control is the no drug treated population (0.2% v/v DMSO liquid NGM). All compound solutions were made in sterile, liquid NGM, with 0.2% v/v DMSO co-solvent due to the fact that Compound 1 is hydrophobic with low solubility in water-based solutions. Each day, at the same time, death scores were determined and three independent trials were conducted, with two technical replicates per trial.

FIG. 4. Compound 1 improves the percentage of highly active C. elegans. To measure locomotory health related parameters the same video recordings that were captured for live/dead scoring were analyzed. The movements of the individual animals were tracked and animals were grouped into cohorts of different activity. This was achieved by determining how much of the animal body moved in a boundary box that encompasses it, in a duration of 30 seconds. Animals were referred to as highly active when their entire body moved out of the box. We plot the percentage of highly active animals in the population relative to day 4 (Day 1, adulthood), and shown for Day 5 and Day8, as well as Days 12-15, which corresponds to approximately the mean lifespan. Results are shown as Mean % of highly active animals and a pairwise t-test was performed to show significant p values from the comparison of control, 0.5 and 10 μM Compound 1 treated nematode activity.

FIG. 5. 0.5 μM Compound 1 improves the proportion of moderately active worms versus inactive worms in the middle stage of lifespan. The movements of the individual animals were tracked and animals were grouped into cohorts of different activity. This was achieved by determining how much of the animal body moved in a boundary box that encompasses it, in a duration of 30 seconds. Animals were referred to as highly active when their entire body moved out of the bounding box. Moderately active animals moved only a portion of their body outside of the bounding box while the body of inactive animals remained within the bounding box. We plot the Mean % of inactive, moderate and highly active animals as a proportion of all living animals on a given day and a graph is shown for each mobility category.

FIG. 6. 10 μM Compound 1 improves the proportion of highly active worms versus inactive worms in the latter stages of lifespan. The movements of the individual animals were tracked and animals were grouped into cohorts of different activity. This was achieved by determining how much of the animal body moved in a boundary box that encompasses it, in a duration of 30 seconds. Animals were referred to as highly active when their entire body moved out of the bounding box. Moderately active animals moved only a portion of their body outside of the bounding box while the body of inactive animals remained within the bounding box. We plot the Mean % of inactive, moderate and highly active animals as a proportion of all living animals on a given day and a graph is shown for each mobility category.

FIG. 7. Compound 1 increases autophagy in Zebrafish larvae. An autophagy reporter zebrafish line was generated by stable expression of the LC3 protein fused to ZsGreen under the control of a skeletal muscle specific promoter. Larvae from outcrossed transgenic zebrafish were raised at 28 C under standard laboratory conditions and have been treated at 48 h post-fertilization in 96 well plates with varying concentrations of Compound 1 at different concentrations as indicated in the figure. After 16 hours of treatment larvae were anesthetized with 0.016% tricaine and imaged with ImageXpress confocal system at 20× magnification (Molecular Devices). Z stack images were captured for each larva and maximal projection images were produced. Ammonium chloride (final concentration 100 mM) was added for an additional 4 h to block lysosomal degradation. Images were acquired again as before. In order to quantify autophagic flux, number of LC3 punctae have been calculated in presence and in absence of ammonium chloride with MetaXpress software (Molecular Devices) and normalized by zebrafish area.

### DETAILED DESCRIPTION

### Definitions

### General Terminology

As used herein, the singular forms “a,” “an” and “the” include plural referents unless the context clearly dictates otherwise. Thus, for example, reference to “a component” or “the component” includes two or more components.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 10th Ed., McGraw Hill Companies Inc., New York (2001). Standard medical terminology used herein has the meaning defined in Stedman's Medical Dictionary, 27th Edition, with veterinary medicine insert.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive.

### General Chemistry Terminology

The descriptions of simple chains like methyl, pentyl, dodecyl are known to the person skilled in the art. For example, methyl is an alkyl derived from methane, containing one carbon atom bonded to three hydrogen atoms-CH3 chemical formula —C3H. Pentyl is a five-carbon alkyl functional group (substituent) with chemical formula —C5H11. Dodecyl is a twelve-carbon alkyl functional group (substituent) with chemical formula —C12H25.

For the avoidance of doubt, more complex chains have been drawn and a number attributed as a quick reference.

In all structures, the dot represents the point of insertion of the chain on the aromatic ring. For example, if in Markush structure A, R2 is 3-methyl-2-buten-1-yl (1h), this corresponds to structure B:

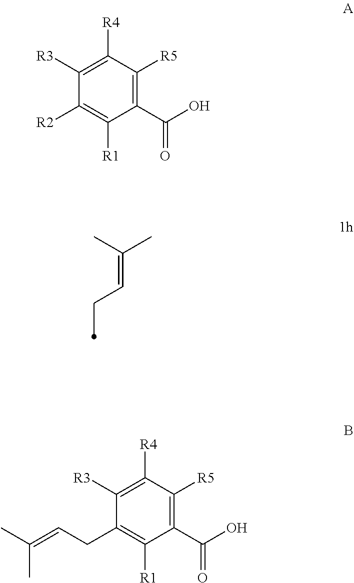

The term C5 isoprenoid unit refers to the following substituents: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p).

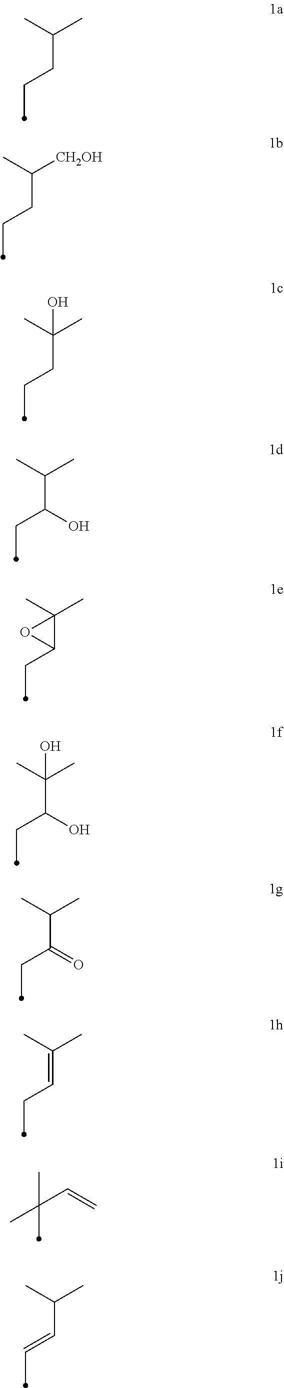

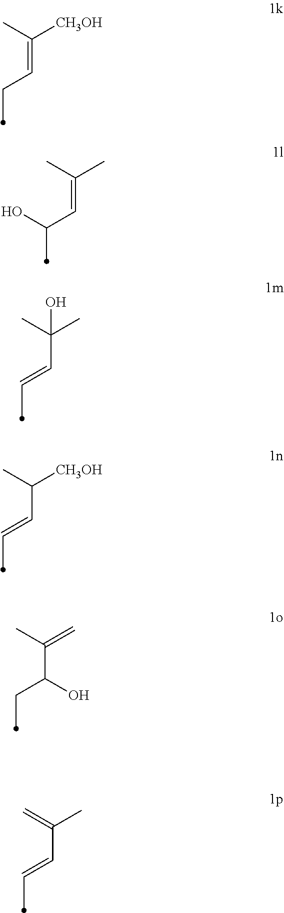

The term C10 isoprenoid unit refers to the following substituents: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o).

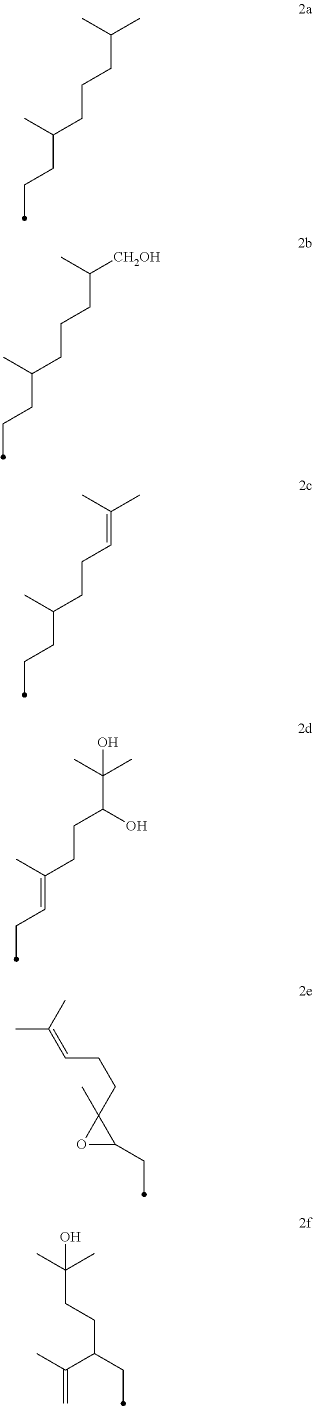

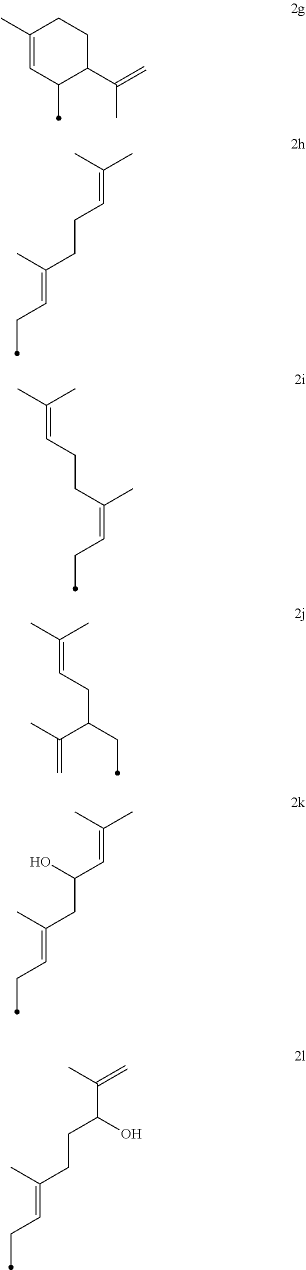

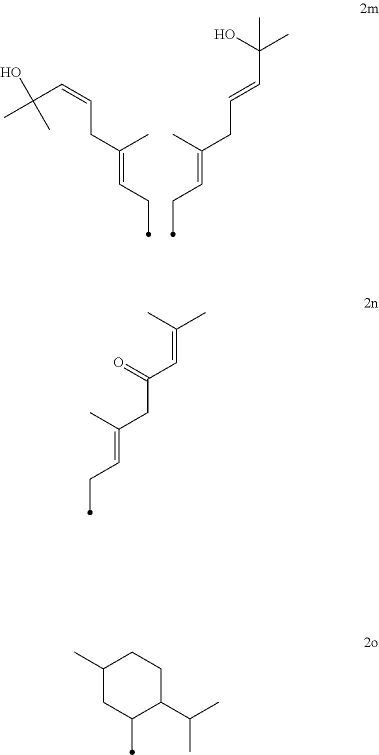

The term C15 isoprenoid unit refers to the following substituents: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d).

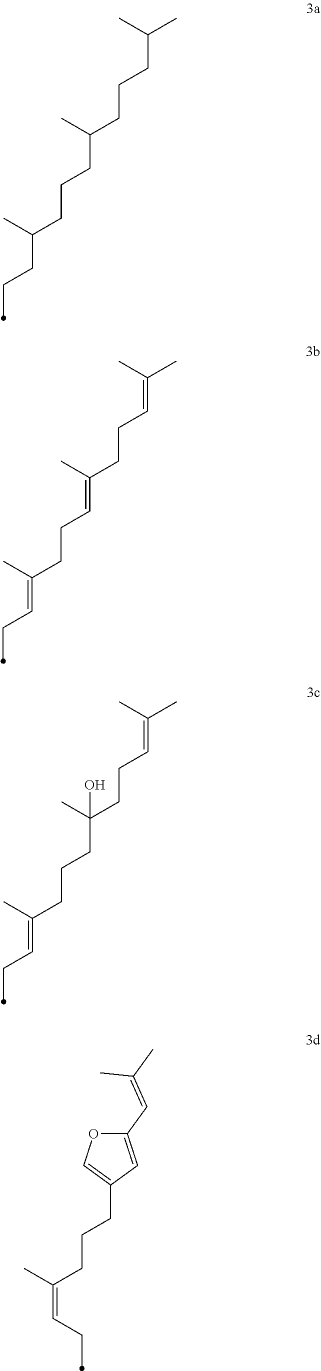

C20 isoprenoid unit refers to the following substituents: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b).

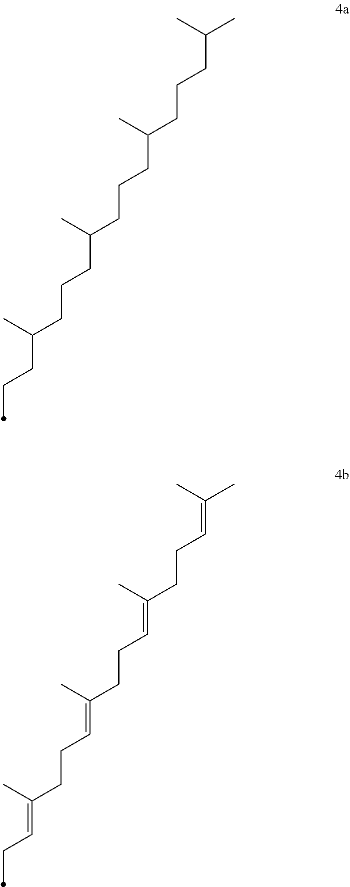

The term “alkyl” refers to a branched or unbranched saturated hydrocarbon chain having from 1 to 20 carbon atoms, or from 1 to 15 carbon atoms, or from 1 to 10 carbon atoms, or from 1 to 7 carbon atoms, or from 1 to 5 carbon atoms, or from 1 to 3 carbon atoms. The alkyl chain may be cyclic, in which case it would be known as “cycloalkyl” group.

Non-limiting examples of branched or unbranched alkyl chains include: methyl, ethyl, n-propyl, iso-propyl, n-butyl, iso-butyl, t-butyl, pentyl, hexyl, heptyl, nonyl, decyl, undecyl, tetradecyl, pentadecyl, heptadecyl, eicosyl.

A non-limiting example of cycloalkyl chain includes: 5-methyl-2-(1-methylethyl)cyclohexyl (2o)

The term “substituted alkyl” refers to:

1) an alkyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)— cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;

One of the oxygen substituents of the alkyl chain can be joined by single bonds to two adjacent carbon atoms of the same alkyl chain, to form an epoxy group, i.e. a three-membered epoxide ring.

Non limiting examples of alkyl chains substituted by hydroxy, alkoxy, and/or acyloxy groups, or containing an epoxy group include: 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 8-hydroxy-3,7-dimethyloctyl (2b), hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p).

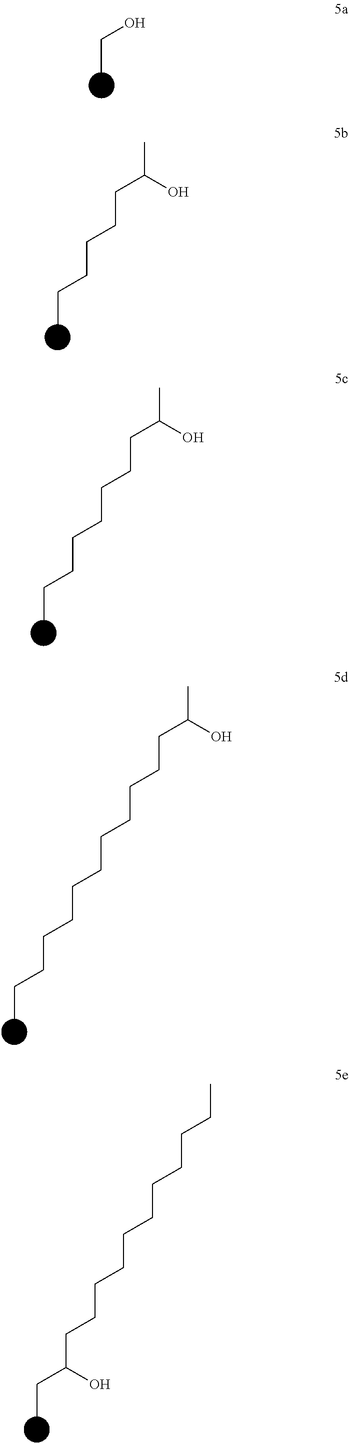

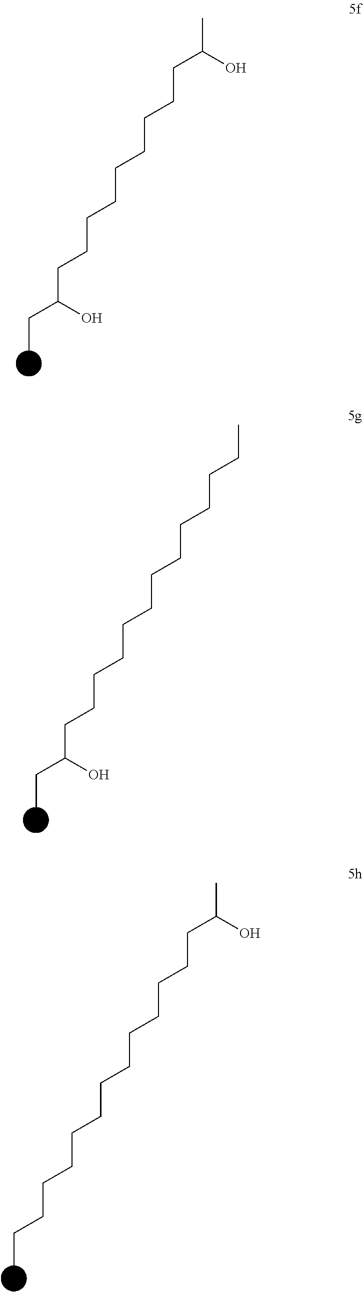 [SMILES: CCC(O)CCCCCCCCCC(C)O ; CCCCCCCCCCCCCC(O)CC ; CCCCCCCCCCCCCCC(C)O]

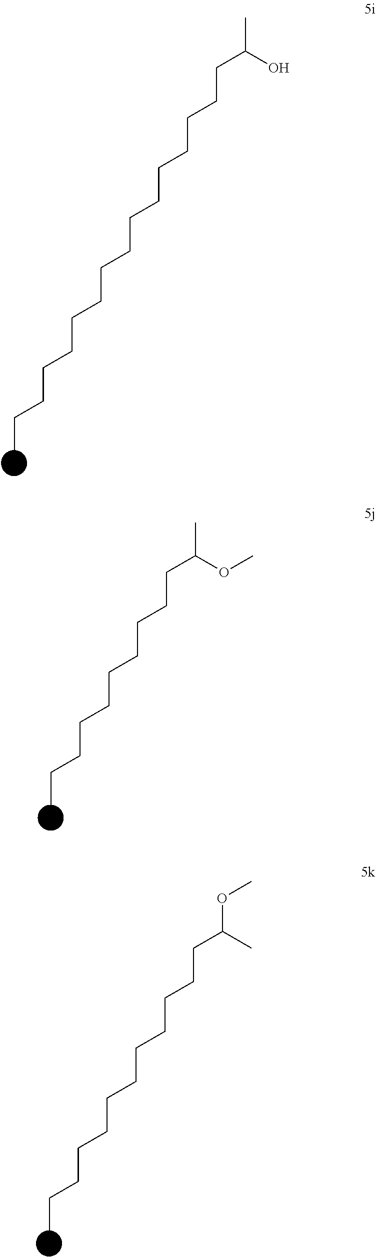 [SMILES: CCCCCCCCCCCCCCCCC(C)O ; CCCCCCCCCCC(C)OC ; CCCCCCCCCCCCC(C)OC]

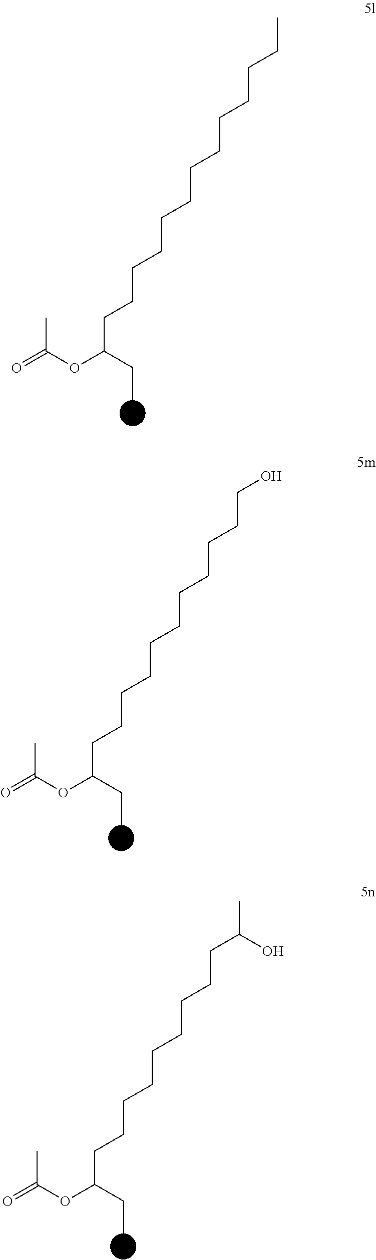 [SMILES: CCCCCCCCCCCCCC(CC)OC(C)=O ; CCC(CCCCCCCCCCCO)OC(C)=O ; CCC(CCCCCCCCCC(C)O)OC(C)=O]

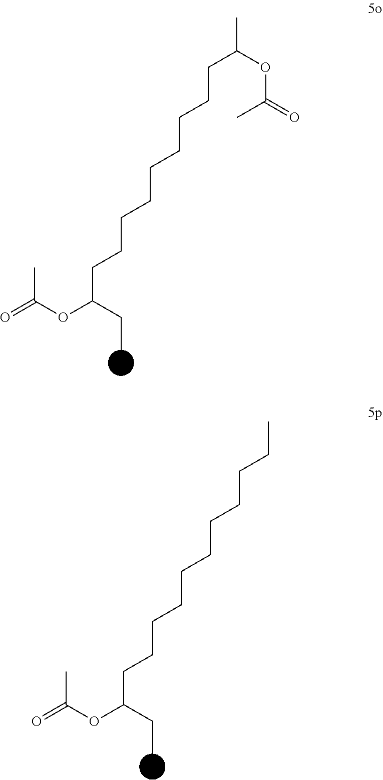 [SMILES: CCC(CCCCCCCCCC(C)OC(C)=O)OC(C)=O ; CCCCCCCCCCCC(CC)OC(C)=O]

Non limiting examples of alkyl chains substituted by aryl/aryloxy groups include: Benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e).

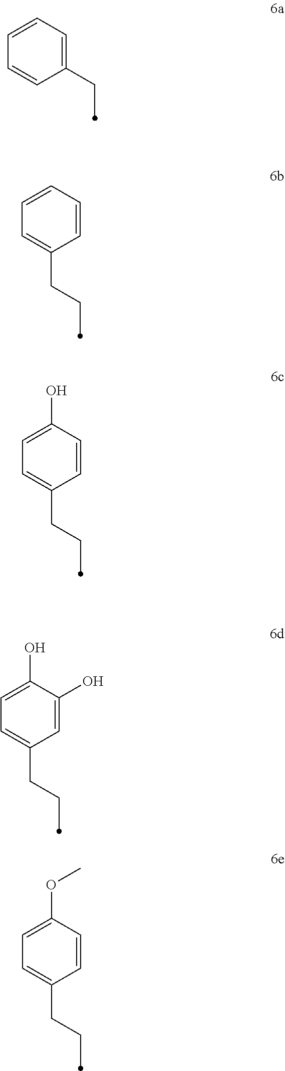

Non limiting examples of alkyl chains substituted by aryl/aryloxy and hydroxy groups include: 2-Hydroxy-2-phenylyethyl (7a), 2-hydroxy-2-(2-hydroxyphenyl)ethyl (7b).

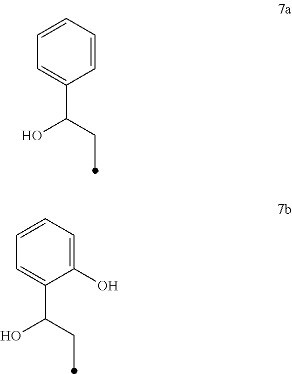

A non-limiting example of alkyl chain substituted by a heterocyclyl group includes: Tetrahydro-6-methyl-2H-pyran-2-yl)methyl (8a).

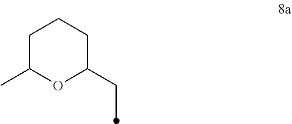

or

2) an alkyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;

or

3) an alkyl chain as defined above that has both 1, 2, 3, 4 or 5 substituents as defined above and is also interrupted by 1-5 atoms (e.g. 1, 2, 3, 4 or 5 atoms) as defined above;

or

4) an alkyl chain as defined above in which one or more of the methylene group is replaced by a carbonyl group to give an oxo group.

Non limiting examples of alkyl chain in which one or more of the methylene group is replaced by a carbonyl group include: 3-methyl-2-oxobutyl (1g), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 2-oxotridecyl (9g). 9a 9b 9c 9d 9e 9f 9g

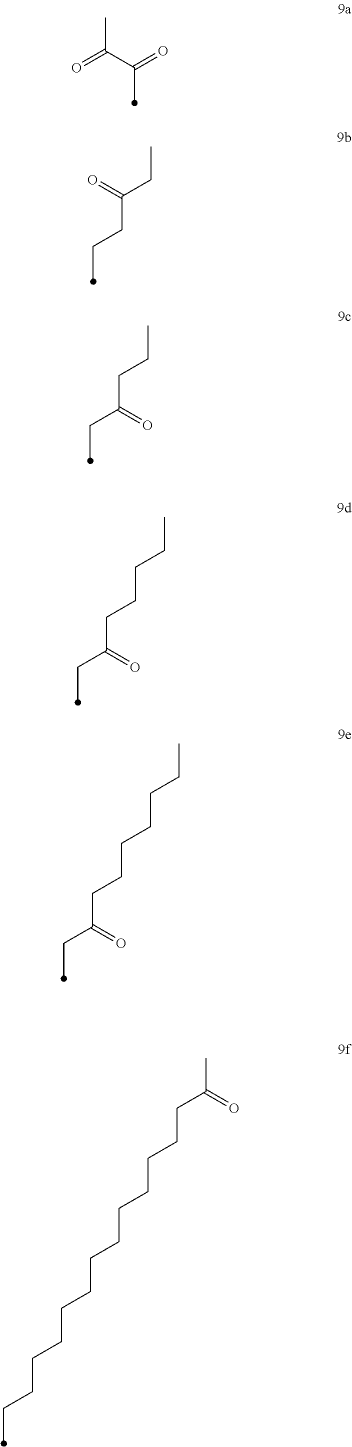

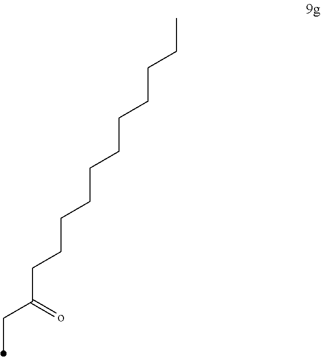

or

5) an alkyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non limiting examples of alkyl chain in which one of the methylene group is replaced by a carbonyl group to give an oxo group, and has a hydroxy substituent include: 1-hydroxy-2-oxopropyl (10a), 13-hydroxy-2-oxotridecyl (10b).

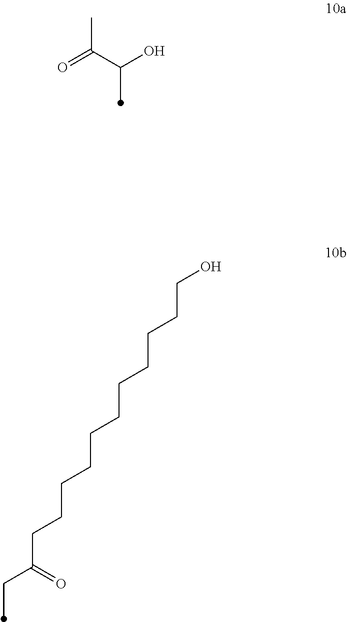

Non limiting examples of alkyl chains substituted by aryl/aryloxy, in which one of the methylene group is replaced by a carbonyl group to give an oxo group includes: 2-(4-hydroxyphenyl)-2-oxoethyl (11a), 2-oxo-2-phenylethyl (11b).

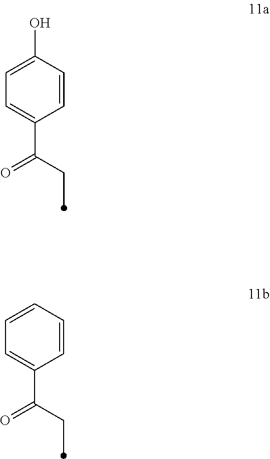

The term “alkenyl” refers to a type of alkyl chain as defined above, in which two atoms of the alkyl chain form a double bond that is not part of an aromatic group. That is, an alkenyl chain contains the pattern R-C(R)═C(R)-R, In one embodiment, R refers to the remaining portions of the alkenyl chain, which may be the same or different. The alkenyl moiety may be branched, straight chain, or cyclic (in which case, it would also be known as a “cycloalkenyl” group).

The term “substituted alkenyl” refers to:

1) an alkenyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)— cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; One of the oxygen substituents of the alkenyl chain can be joined by single bonds to two adjacent carbon atoms of the same alkenyl chain, to form an epoxy group, i.e. a three-membered epoxide ring.

or
2) an alkenyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) an alkenyl chain as defined above that has both 1, 2, 3, 4 or 5 substituents as defined above and is also interrupted by 1-5 atoms (e.g. 1, 2, 3, 4 or 5 atoms) as defined above; or
4) an alkenyl chain as defined above in which one or more of the methylene group is replaced by a carbonyl group to give an oxo group.
   or
5) an alkenyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non-limiting examples of alkenyl chains include: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 3,7-dimethyloct-6-en-1-yl (citronellyl) (2c), ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl(12g).

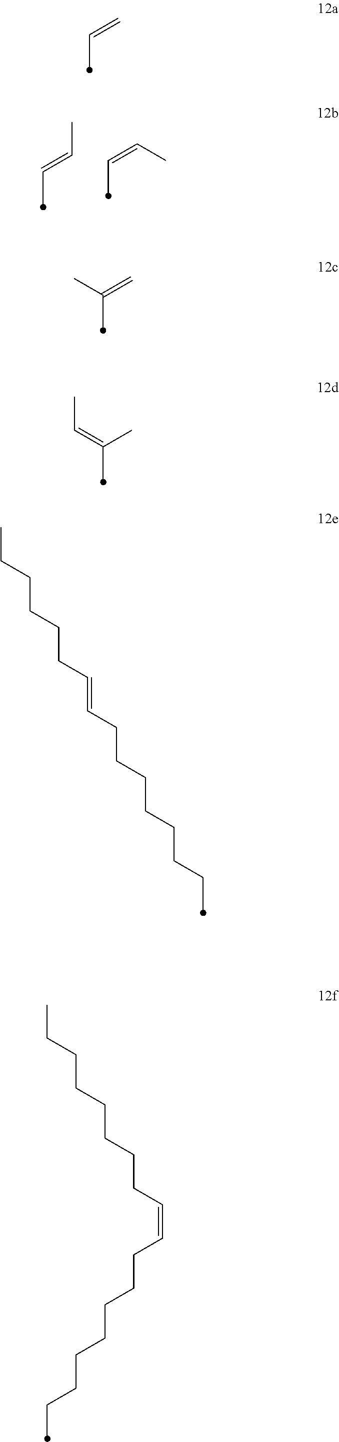

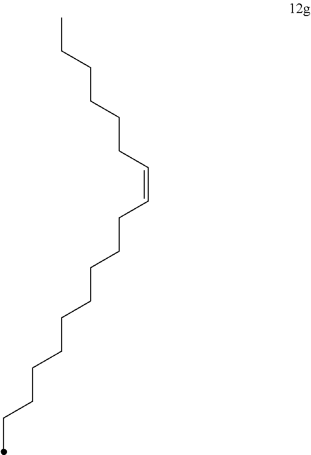

Non limiting examples of alkenyl chains substituted by hydroxy, and/or acyloxy groups, or containing an epoxy group include: 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 10-(acetyloxy)-8-pentadecenyl (13a).

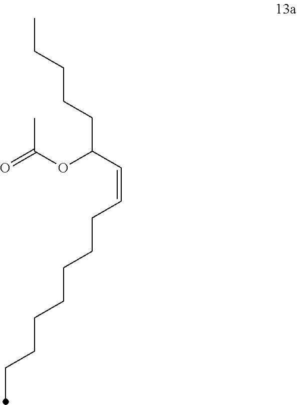

A non-limiting example of alkenyl chain substituted by an aryl group includes: 2-phenylethenyl (14a).

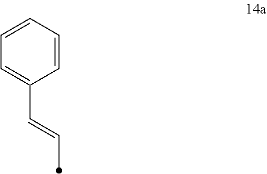

A non-limiting example of an alkenyl chain where one of the methylene is replaced by an oxo group includes: 1-hydroxymethylene-2-oxopropyl (15a).

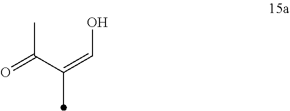

The term “alkynyl” refers to a type of alkyl chain as defined above in which two atoms of the alkyl chain form a triple bond. That is, an alkynyl chain contains the pattern R-C≡C-R, In one embodiment, R refers to the remaining portions of the alkynyl chain, which may be the same or different. Non-limiting examples of an alkynyl chain include —C≡CH, —C≡C—CH3 and —C ≡C—CH2-CH3. The “R” portion of the alkynyl moiety may be branched, straight chain, or cyclic. Alkynyl chains can be optionally substituted.

The term “substituted alkynyl” refers to:
1) an alkynyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)— cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; One of the oxygen substituents of the alkynyl chain can be joined by single bonds to two adjacent carbon atoms of the same alkynyl chain, to form an epoxy group, i.e. a three-membered epoxide ring.
   or
2) an alkynyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) an alkynyl chain as defined above that has both 1, 2, 3, 4 or 5 substituents as defined above and is also interrupted by 1-5 atoms (e.g. 1, 2, 3, 4 or 5 atoms) as defined above; or
4) an alkynyl chain as defined above in which one or more of the methylene group is replaced by a carbonyl group to give an oxo group.
   or
5) an alkynyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

The term polyalkenyl refers to a chain in which more than one pair of atoms of the alkyl chain form a double bond that is not part of an aromatic group. That is, a polyalkenyl chain contains from 2 to 8 R—C(R)═C(R)-R patterns, In one embodiment, R refers to the remaining portions of the alkenyl chain, which may be the same or different. The polyalkenyl moiety may be branched, or straight chain.

Non-limiting examples of polyalkenyl chains include: 3-methyl-1,3-butadienyl (1p), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), (2E,6E)-3,7,11-trimethyl-2,6,10-dodecatrien-1-yl (farnesyl) (3b), (2E,6E,10E)-3,7,11,15-tetramethylhexadeca-2,6,10,14-tetraen-1-yl (geranylgeranyl) (4b), 8,11-heptadecadien-1-yl (16a).

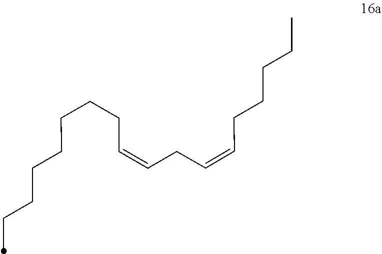

The polyalkenyl moiety containing two double bonds may be cyclic (in which case, it would also be known as a “cyclodialkenyl” group). Non limiting example of cyclodialkenyl groups include cyclopentadiene and cyclohexadiene groups. Polyalkenyl chains can be optionally substituted.

The term “substituted polyalkenyl” refers to:
1) a polyalkenyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; One of the oxygen substituent of the polyalkenyl chain can be joined by single bonds to two adjacent carbon atoms of the same polyalkenyl chain, to form an epoxy group, i.e. a three-membered epoxide ring. One of the oxygen substituent of the polyalkenyl chain can from a five-membered aromatic ring with four carbon atoms of the same chain, resulting in. a furan ring.
   or
2) a polyalkenyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) a polyalkenyl chain as defined above that has both 1, 2, 3, 4 or 5 substituents as defined above and is also interrupted by 1-5 atoms (e.g. 1, 2, 3, 4 or 5 atoms) as defined above; or
4) a polyalkenyl chain as defined above in which one or more of the methylene group is replaced by a carbonyl group to give an oxo group.
   or
5) a polyalkenyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non limiting examples of polyalkenyl chains substituted by a hydroxy group, or containing a furan ring include: 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d).

Non limiting examples of linear or cyclized polyalkenyl chains in which one of the methylene group is replaced by a carbonyl group to give an oxo group, include: 3,7-dimethyl-5-oxo-2,6-octadienyl (2n), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c).

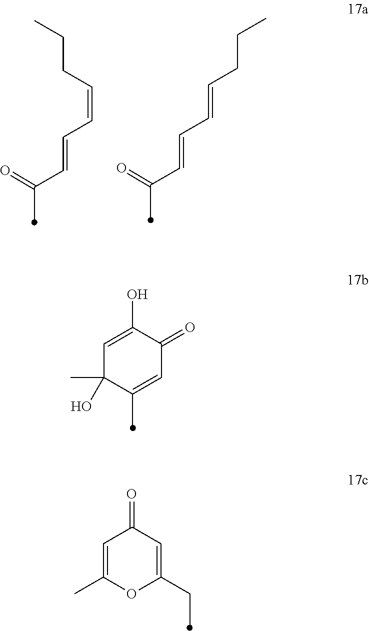

A non limiting example of a polyalkenyl chain substituted by an aryloxy group includes 8-(3,4-Dihydroxyphenyl)-4,7-octadien-1-yl (18a).

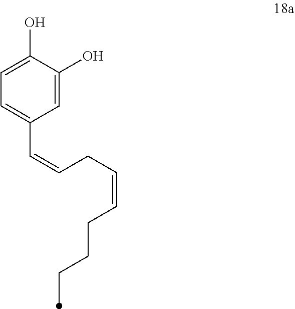

The term polyalkynyl refers to a chain in which more than one pair of atoms of the alkyl chain form a triple bond. That is, a polyalkynyl chain contains from 2 to 8 R-C≡C-R patterns, In one embodiment, R refers to the remaining portions of the alkynyl chain, which may be the same or different. Non-limiting example of a polyalkynyl chain include —CH2-CH2-C≡C—C ≡CH. The “R” portion of the polyalkynyl moiety may be branched, straight chain, or cyclic.

The term “substituted polyalkynyl” refers to:
1) a polyalkynyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
2) a polyalkynyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) a polyalkynyl chain as defined above that has both 1, 2, 3, 4 or 5 substituents as defined above and is also interrupted by 1-5 atoms (e.g. 1, 2, 3, 4 or 5 atoms) as defined above; or
4) a polyalkynyl chain as defined above in which one or more of the methylene group is replaced by a carbonyl group to give an oxo group.
   or
5) a polyalkynyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

The term “polyunsaturated” refers to a chain in which at least one pair of atoms of the alkyl chain form a double bond and one pair of atoms of the alkyl chain form a triple bond. That is, a polyunsaturated chain contains both R—C(R)═C(R)-R and R-C≡C-R patterns, In one embodiment, R refers to the remaining portions of the polyunsaturated chain, which may be the same or different and the total number of unsaturated bonds may vary from 2 to 8. Non-limiting examples this type of polyunsaturated chain include —CH2-CH═CH—C≡CH. The “R” portion of the polyunsaturated moiety may be branched, straight chain, or cyclic.

The term “substituted polyunsaturated” refers to:
1) a polyunsaturated chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
2) a polyunsaturated chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2;

or

3) a polyunsaturated chain as defined above that has both 1, 2, 3, 4 or 5 substituents as defined above and is also interrupted by 1-5 atoms (e.g. 1, 2, 3, 4 or 5 atoms) as defined above;

or

4) a polyunsaturated chain as defined above in which one or more of the methylene group is replaced by a carbonyl group to give an oxo group.

or

5) a polyunsaturated chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system. As used herein, the term “ring system” refers to two or more rings, In one embodiment, two or more of the rings are fused. The term “fused” refers to structures in which two or more rings share one or more bonds.

The term “halogen” may refer to a fluorine atom, a chlorine atom, a bromine atom or an iodine atom.

The term “glycoside” refers to a compound in which at least one sugar is bound to another functional group via a glycosidic bond. Typically the glycosidic chain can comprise 1 to 4 sugar units.

The term “glycosidic bond” refers to a bond formed between the hemiacetal or hemiketal group of a sugar and the chemical group of a compound. The chemical group can be —OH (O-glycoside), or —CR1R2R3 (C-glycoside).

The terms “acylated O-glycoside” and “acylated C-glycoside” refer to a compound in which at least one hydroxyl of the glycosidic chain is esterified by an organic acid. Typical examples or organic acid may comprise acetic, substituted benzoic, cinnamic (caffeic, ferulic, p-coumaric), and/or phenylpropanoic (dihydrocaffeic) acids.

The terms “sulfated O-glycoside” and “sulfated C-glycoside” refer to a compound in which at least one hydroxyl of the glycosidic chain is esterified by sulfuric acid.

The term “methylene dioxy” may refer to functional group with the structural formula R-O—CH2-O-R′, connected to the rest of a molecule by two chemical bonds.

The term “analogue” as used herein is understood to refer to a compound having a structure similar to that of another one but differing from it in respect of a certain component. A “derivative” is a compound that can be imagined to arise or is actually be synthesized from a parent compound by replacement of one or more atoms with another atom or group of atoms.

### Compound or Composition

It is understood that according to certain embodiments, the compound of the invention or composition thereof may be a nutraceutical composition, pharmaceutical composition, functional food, functional nutrition product, medical food, medical nutrition product, or a dietary supplement.

The terms “nutraceutical” combines the words “nutrition” and “pharmaceutical”. It is a food or food product that provides health and medical benefits, including the prevention and treatment of a condition, disorder, or disease. A nutraceutical is a product isolated or purified from foods that is generally sold in medicinal forms not usually associated with food. A nutraceutical is demonstrated to have a physiological benefit or provide protection against a condition, disorder, or disease. Such products may range from isolated nutrients, dietary supplements and specific diets to genetically engineered foods, herbal products, and processed foods such as cereals, soups, and beverages.

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Moreover, a multi-vitamin and mineral supplement may be added to nutraceutical compositions of the invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions of the invention may be in any galenic form that is suitable for administering to the body, especially in any form that is conventional for oral administration, e.g. in solid forms such as food or feed, food or feed premix, fortified food or feed, tablets, pills, granules, dragees, capsules and effervescent formulations such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or lignin sulfonate. Examples for other application forms are those for transdermal, parenteral or injectable administration. The dietary and pharmaceutical compositions may be in the form of controlled (delayed) release formulations.

Beverages encompass non-alcoholic and alcoholic drinks as well as liquid preparations to be added to drinking water and liquid food. Non-alcoholic drinks are e.g. soft drinks, sports drinks, fruit juices, teas and milk-based drinks. Liquid foods are e.g. soups and dairy products. The nutraceutical composition comprising the compound of the invention may be added to a soft drink, an energy bar, or a candy.

If the nutraceutical composition is a pharmaceutical formulation and the composition further contains pharmaceutically acceptable excipients, diluents or adjuvants then standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

“Functional food”, “functional nutrition product”, “medical food” and “medical nutrition product” relate to any healthy food claimed to have a health-promoting or disease-preventing property beyond the basic function of supplying nutrients. The general category of functional foods includes processed food or foods fortified with health-promoting additives, like “vitamin-enriched” products.

The terms “food,” “food product” and “food composition” or “diet product” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

A dietary supplement, also known as food supplement or nutritional supplement, is a preparation intended to supplement the diet and provide nutrients, such as vitamins, minerals, fibre, fatty acids, or amino acids that may be missing or may not be consumed in sufficient quantities in a person's diet. Some countries define dietary supplements as foods, while in others they are defined as drugs or natural health products. Supplements containing vitamins or dietary minerals are included as a category of food in the Codex Alimentarius, a collection of internationally recognized standards, codes of practice, guidelines and other recommendations relating to foods, food production and food safety. These texts are drawn up by the Codex Alimentarius Commission, an organization that is sponsored by the Food and Agriculture Organization of the United Nations (FAO) and the World Health Organization (WHO).

Compositions intended for an animal, include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the pet food composition ranges from less than 1% to about 15%, and in one aspect, from about 5% to about 10%.

The moisture content can vary depending on the nature of the pet food composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for companion animals.

### Methods

“Prevention” or “preventing” includes reduction of risk and/or severity of a condition, disorder, or disease.

The terms “treatment,” “treating,”, “treat”, “attenuate” and “alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in a subject not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

The term “subject” or “individual” means any animal, including a human, that could benefit from one or more of the compounds, compositions or methods disclosed herein. Generally, the subject is a human or an avian, bovine, canine, equine, feline, hircine, lupine, murine, ovine or porcine animal. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the subject is a human or a companion animal such as a dog or cat. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly subjects. For other animals, an “older adult” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An elderly cat or dog has an age from birth of at least about 7 years.

As used herein, an “effective amount” is an amount that prevents a deficiency, treats a disorder, condition, or disease in a subject or, more generally, reduces symptoms, manages progression of the diseases or provides a nutritional, physiological, or medical benefit to the subject. The relative terms “improved,” “increased,” “enhanced” and the like refer to the effects of the composition disclosed herein relative to a composition lacking one or more ingredients and/or having a different amount of one or more ingredients, but otherwise identical.

The compound of the invention or composition thereof is preferably administered by oral administration. In some embodiments, the compound of the invention or composition thereof may be administered by intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The composition of the invention can have an acute effect that can be seen in less than one month. Additionally or alternatively, the composition can have a longterm effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day. In one embodiment, a single dose is not less than about 100 mg. In one embodiment, a single dose is not more than about 1000 mg. In one embodiment, a single dose is between about 100 mg and about 1000 mg.

For humans, some embodiments comprise administering an amount of the composition that provides 0.1 mg to 50 mg CBDA/kg of body weight of the human, preferably 1 to 25 mg per kg of body weight of the human. In some embodiments, at least a portion of the compound having general formula I is isolated from natural plant sources.

As used herein, an “AMPK activator” refers to a compound that either increases the phosphorylation of downstream substrates of (phosphorylated or not) AMPK, and/or that increases the phosphorylation of AMPK.

As used herein, a “direct AMPK activator” refers to a compound that activates AMPK via direct interaction with at least one of its subunits. As used herein, “AMPK-related diseases” includes pathologic or pathogenomic conditions in which the activation of AMPK provides a salutary effect. Examples of such diseases or conditions include aging. In addition, “AMPK-related conditions” include conditions where the activation of AMPK improves the condition associated with the primary “AMPK-related disease”.

In an embodiment, at least a portion of the one or more cells are part of at least one body part selected from the group consisting of liver, kidney, brain and skeletal muscle.

In another embodiment, metabolic fatigue comprises lack of energy, in particular physical energy, lack of vitality or weakness.

In some embodiments, the methods comprise identifying the individual as having the condition or being at risk of the condition before the administration.

The methods disclosed herein can be useful for treating conditions involving stress injury to mitochondria, which injury may be manifest in any of a number of ways including, but not limited to, mitochondrial disease.

Mitochondrial diseases are the result of either inherited or spontaneous mutations in mitochondrial DNA or nuclear DNA which lead to altered functions of the proteins or RNA molecules that normally reside in mitochondria. Problems with mitochondrial function, however, may only affect certain tissues as a result of factors occurring during development and growth that are not yet fully understood. Even when tissue-specific isoforms of mitochondrial proteins are considered, it is difficult to explain the variable patterns of affected organ systems in the mitochondrial disease syndromes seen clinically.

Mitochondrial diseases result from failures of the mitochondria, specialized compartments present in every cell of the body except red blood cells. Mitochondria are responsible for creating more than 90% of the energy needed by the body to sustain life and support growth. When they fail, less and less energy is generated within the cell. Cell injury and even cell death follow. If this process is repeated throughout the body, whole systems begin to fail, and the life of the person in whom this is happening is severely compromised. Mitochondrial diseases primarily affect children, but adult onset is becoming more recognized.

Diseases of the mitochondria appear to cause the most damage to cells of the brain, heart, liver, skeletal muscles, kidney, and the endocrine and respiratory systems. Many symptoms in mitochondrial disorders are non-specific. The symptoms may also show an episodic course, with periodic exacerbations. The episodic condition of migraine, as well as myalgia, gastrointestinal symptoms, tinnitus, depression, chronic fatigue have been mentioned among the various manifestations of mitochondrial disorders in review papers on mitochondrial medicine. In patients with mitochondrial disorders, clinical symptomatology typically occurs at times of higher energy demand associated with physiological stressors, such as illness, fasting, over-exercise, and environmental temperature extremes. Furthermore, psychological stressors also frequently trigger symptomatology, presumably due to higher brain energy demands for which the patient is unable to match with sufficient ATP production.

Depending on which cells are affected, symptoms may include loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

Mitochondrial diseases include, without limitation, Alper's disease; Barth syndrome; beta-oxidation defects; carnitine deficiency; carnitine-acyl-carnitine deficiency; chronic progressive external ophthalmoplegia syndrome; co-enzyme Q10 deficiency; Complex I deficiency; Complex II deficiency; Complex III deficiency; Complex IV deficiency; Complex V deficiency; CPT I deficiency; CPT II deficiency; creatine deficiency syndrome; cytochrome c oxidase deficiency; glutaric aciduria type II; Kearns-Sayre syndrome; lactic acidosis; LCHAD (long-chain acyl-CoA dehydrogenase deficiency); Leber's hereditary optic neuropathy; Leigh disease; lethal infantile cardiomyopathy; Luft disease; MAD (medium-chain acyl-CoA dehydrogenase deficiency); mitochondrial cytopathy; mitochondrial DNA depletion; mitochondrial encephalomyopathy, lactic acidosis, and stroke-like symptoms; mitochondrial encephalopathy; mitochondrial myopathy; mitochondrial recessive ataxia syndrome; muscular dystrophies, myoclonic epilepsy and ragged-red fiber disease; myoneurogenic gastrointestinal encephalopathy; neuropathy, ataxia, retinitis pigmentosa, and ptosis; Pearson syndrome; POLG mutations; pyruvate carboxylase deficiency; pyruvate dehydrogenase deficiency; SCHAD (short-chain acyl-CoA dehydrogenase deficiency); and very long-chain acyl-CoA dehydrogenase deficiency.

The present combination of ingredient is also effective in improving healthspan. Moreover, increasing mean lifespan is an indication for improved healthspan as shown in Martineau C N, Brown A E X, Laurent P (2020) PLoS Comput Biol 16(7).

The stress that is treated or prevented can be early-life stress, i.e., stress experienced while under the age of five years from birth. Early-life stress has been reported to have a significant detrimental effect on cognitive performance, including psychological parameters such as increased rates of or susceptibility to depression, anxiety, and abnormal risk-taking behavior. Increased rates of attention-deficit/hyperactivity disorder (ADHD), post-traumatic stress disorder (PTSD), and major depression have been reported in individuals having experienced early-life stress.

Another aspect of the present disclosure is a method of delaying off-set of metabolic decline, maintaining muscle mass, decreasing oxidative stress, maintaining immune function and/or maintaining cognitive function in a healthy older adult.

As used herein, “cognitive function” refers to any mental process that involves symbolic operations, e.g., perception, memory, attention, speech comprehension, speech generation, reading comprehension, creation of imagery, learning, and reasoning, preferably at least memory. Methods for measuring cognitive function are well-known and can include, for example, individual or battery tests for any aspect of cognitive function. One such test is the Prudhoe Cognitive Function Test by Margallo-Lana et al. (2003) J. Intellect. Disability Res. 47:488-492. Another such test is the Mini Mental State Exam (MMSE), which is designed to assess orientation to time and place, registration, attention and calculation, recall, language use and comprehension, repetition, and complex commands. As used herein, a “cognitive disorder” refers to any condition that impairs cognitive function. Non-limiting examples of a cognitive disorder include delirium, dementia, learning disorder, attention deficit disorder (ADD), and attention deficit hyperactivity disorder (ADHD). A “stress-induced or stress-related cognitive dysfunction” refers to a disturbance in cognitive function that is induced or related to stress.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the claims. Numerous specific details are set forth in the description in order to provide a thorough understanding of the present invention. The present invention may be practiced without some or all of these specific details. In other instances, well known methods and protocols have not been described in detail, in order not to unnecessarily obscure the present invention.

### EXAMPLES

### Example 1 Experiments on Thermotolerance of C. elegans after a Heat-Shock of 37 C for 4 Hours

It has been reported that exposing C. elegans to high temperatures increases the molecular stress and reduces animal lifespan. Interestingly, the resistance to heat stress is one of the hallmarks of longevity in C. elegans, and has been used in the field for some time as an accelerated lifespan test. Importantly, this has been used to screen and evaluate the effectiveness of anti-ageing compounds. We monitored the survival of C. elegans after a thermal stress to determine whether Compound 1 could improve the ageing of these animals and at what concentrations this may occur. A microfluidic Infinity System was used to assess the survival of the C. elegans treated with varying concentrations of Compound 1 and vehicle (control) worms following a heat stress challenge. To achieve this, worms were maintained at their normal temperature (20 C) and then treated for 48-hour incubation with Compound 1 starting on adult day 1, followed by 4-hour heat challenge at 37 C, followed by 15 mins recovery and then death was recorded.

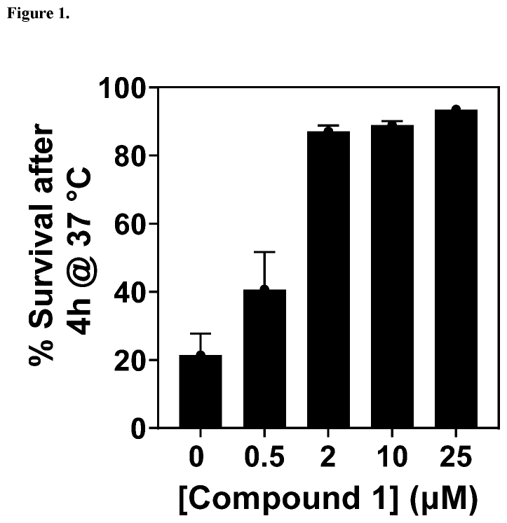

In the absence of any treatment (control), only around 20% of WT worms survive this heat shock. In contrast, in the presence of varying concentrations of Compound 1, there was a significant decrease in the percentage of WT C. elegans that die due to this heat shock. As shown in FIG. 1, at 0.5 μM there was a small improvement in survival, but at the subsequent concentrations, there was almost a complete prevention of C. elegans death after exposure to heat-stress. The ability of Compound 1 to improve survival of C. elegans after a heat shock, suggests that Compound 1 has the possibility to increase lifespan in C. elegans.

### Example 2: Experiments on Lifespan of C elegans in an AMPK-Dependent Manner

To determine whether Compound 1 can extend lifespan, C. elegans were treated with 0.5 μM or 10 μM Compound 1 from day 1 of adulthood. The microfluidic Infinity System was used to assess the lifespan of Compound 1-treated and non-treated control worms. 60-70 synchronized day 1 adult animals were loaded in each Infinity chip and treated with 0.5 μM or 10 μM of Compound 1, lifelong, with daily fresh doses of 20 mg/mL of OP50 bacteria at 20 C. Control is the no-drug-treated population, maintained in 0.2% v/v DMSO liquid NGM. All compound solutions were made in sterile, liquid NGM, with 0.2% v/v DMSO co-solvent because both samples are hydrophobic with low solubility in water-based solutions. Each day, at the same time death scores were determined using the Infinity Code. Three independent trials with two technical replicates per trial were conducted.

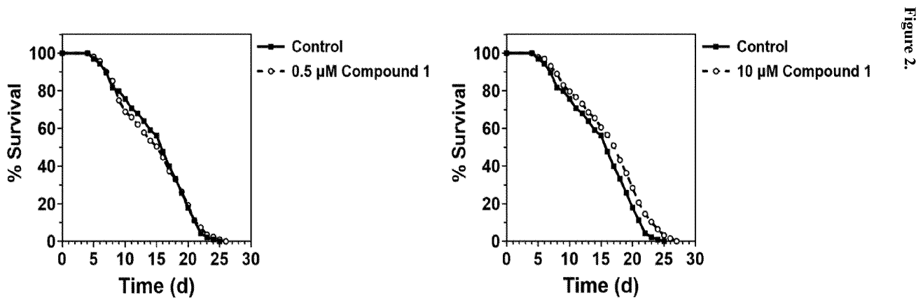

As shown in FIG. 2, in contrast to 0.5 μM Compound 1, 10 μM of Compound 1 significantly extended mean and maximum lifespan. As shown in Table 1, the mean lifespan extension was around 11% with the maximum lifespan extension of 2 additional days.

TABLE 1
Lifespan data of wild-type C. elegans treated with 0.5
μM and 10 μM Compound 1 and untreated controls.

| | Com- | | | % | | |
|---|---|---|---|---|---|---|
| | pound | Sample | Mean | lifespan | Maximum | p |
| | 1 | size* | lifespan | extension | lifespan | Value** |
| Trial 1 | Control | 162 | 15.9 ± 5.1 | | 25 | |
| | 0.5 μM | 168 | 16.6 ± 5.8 | 4.4 | 25 | 0.8136 |
| | 10 μM | 179 | 17.5 ± 5.8 | 10 | 27 | 0.0003 |
| Trial 2 | Control | 144 | 15.3 ± 5.4 | | 25 | |
| | 0.5 μM | 178 | 15.2 ± 5.7 | 0 | 26 | 0.5246 |
| | 10 μM | 183 | 17.1 ± 5.9 | 11.8 | 27 | 0.0091 |
| Trial 3 | Control | 174 | 15.0 ± 5.2 | | 25 | |
| | 0.5 μM | 108 | 15.0 ± 5.4 | 0 | 26 | 0.5703 |
| | 10 μM | 90 | 18.8 ± 6.0 | 25 | 26 | 0.0003 |
| Pooled | Control | 485 | 15.1 ± 5.2 | | 25 | |
| | 0.5 μM | 454 | 15.3 ± 5.4 | 1.3 | 26 | 0.2889 |
| | 10 μM | 452 | 16.8 ± 5.6 | 11.3 | 27 | <0.0001 |

*Values of sample size come from pooling two technical replicates except for the Pooled rows, where data is pooled from all Trials 1 to 3.
*Lifespan curves were compared with the log-rank test.

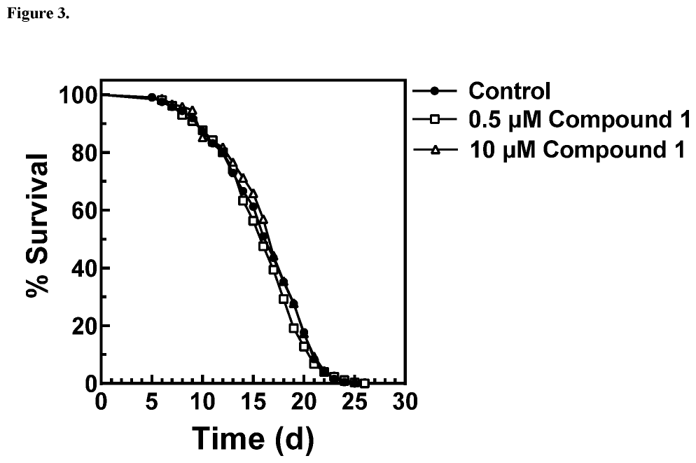

We tested the ability of Compound 1 to extend lifespan in C elegans which have the AMP-activated protein kinase (AMPK) α2 subunit isoform deletion. This mutant strain (aak-2) is commonly used in lifespan studies to look at the dependence on activation of AMPK for mediating the lifespan and other effects of various compounds. Importantly, the aak-2 worm strain itself did not display any differences in the mean or maximum lifespan in this study. Interestingly, the ability of Compound 1 extend lifespan in wild-type C. elegans is completely lost in the aak-2 worms (FIG. 3), demonstrating that Compound 1 works through activating AMPK and leading to lifespan extension. This is a very interesting finding to demonstrate the mechanism of action of Compound 1. The full lifespan parameters are shown in Table 2.

TABLE 2
Lifespan data of AMPKα2 knockout C. elegans (aak-2) treated with
0.5 μM and 10 μM Compound 1 and untreated controls.

| | Com- | | | % | | |
|---|---|---|---|---|---|---|
| | pound | Sample | Mean | lifespan | Maximum | p |
| | 1 | size* | lifespan | extension | lifespan | Value** |
| Trial 1 | Control | 180 | 16.1 ± 4.3 | | 23 | |
| | 0.5 μM | 183 | 16.0 ± 4.2 | 0 | 25 | 0.2904 |
| | 10 μM | 181 | 14.9 ± 3.8 | −7.5 | 23 | 0.159 |
| Trial 2 | Control | 102 | 16.9 ± 4.1 | | 25 | |
| | 0.5 μM | 195 | 16.1 ± 4.0 | −4.7 | 26 | 0.0101 |
| | 10 μM | 246 | 16.2 ± 4.7 | −4.1 | 26 | 0.2857 |
| Trial 3 | Control | 167 | 16.2 ± 4.3 | | 24 | |
| | 0.5 μM | 224 | 15.6 ± 4.4 | −3.7 | 25 | 0.1354 |
| | 10 μM | 156 | 16.8 ± 3.5 | 3.8 | 24 | 0.6077 |
| Pooled | Control | 449 | 16.1 ± 4.4 | | 25 | |
| | 0.5 μM | 602 | 15.9 ± 4.2 | −1.2 | 26 | 0.105 |
| | 10 μM | 583 | 16.1 ± 4.2 | 0 | 26 | 0.3778 |

*Values of sample size come from pooling two technical replicates except for the Pooled rows, where data is pooled from all Trials 1 to 3.
*Lifespan curves were compared with the log-rank test.

### Example 3: Compound 1 Improves the Percentage of Highly Active C. elegans

It is possible to measure the mobility of C. elegans and monitor this throughout the lifespan of the worms. As with many species, C. elegans display an age-related decline in mobility. It has been demonstrated that compounds which improve lifespan, can also improve the mobility of worms throughout lifespan or at specific stages of life. Therefore, we tested whether Compound 1 could improve the mobility of C. elegans throughout their lifespan. To measure locomotory health related parameters the same video recordings that were captured for live/dead scoring were analyzed using the Infinity Code. The movements of the individual animals were tracked and animals were grouped into cohorts of different activity. The cohort analysis involved determining how much of the animal body moved in the boundary box that encompasses it, in a duration of 30 seconds. Animals were referred to as highly active when their entire body moved out of the bounding box. Moderately active animals moved only a portion of their body outside of the bounding box while the body of inactive animals remained within the bounding box.

It is important to note that Day 5 corresponds to day 2 of adulthood and Days 12-15 corresponds to approximately the mean lifespan.

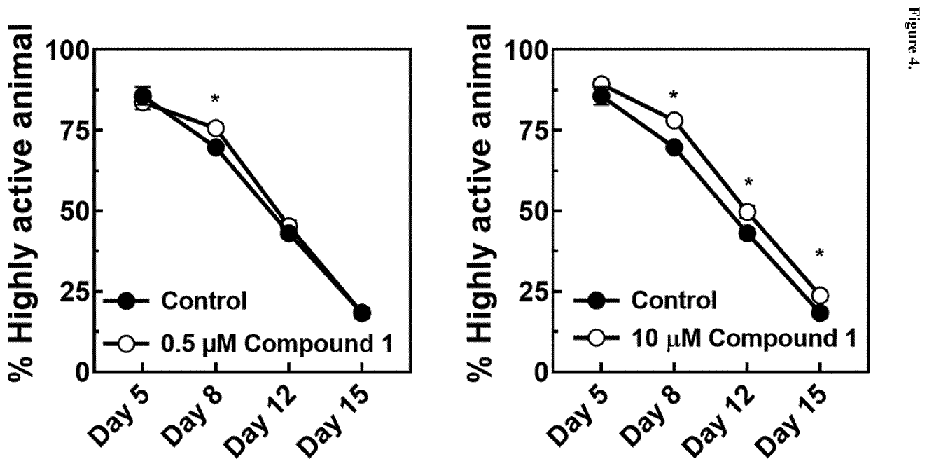

Within the field of ageing it is common to display the mobility of worms by showing the percentage of highly active animals at Day 5, 8, 12 and 15 and compare treated and untreated worms. As shown in FIG. 4, we show that 0.5 μM increases the percentage of highly active animals at Day 8 only, whereas 10 μM increases the percentage of highly active animals at Days 8, 12 and 15. This suggests that Compound 1 can improve the mobility of worms and increase the number of highly active worms at different stages of life. Table 3 shows the exact numbers that were used to generate FIG. 4, with the p values.

TABLE 3
Mean % of highly active animals from the comparison of
control, 0.5 and 10 μM Compound 1 treated
C. elegans. The data is presented in a graph shown
in FIG. 4. p value shown is from a pairwise t-test.

| | | | Mean % of Highly | | |
|---|---|---|---|---|---|
| | Compound 1 | | Active animals | SD | p Value |
| Day 5 | Control | | 85.7 | 11.6 | |
| | 0.5 | μM | 83.7 | 9.1 | 0.5708 |
| | 10 | μM | 89.3 | 6.1 | 0.2498 |
| Day 8 | Control | | 69.7 | 5 | |
| | 0.5 | μM | 75.7 | 5.6 | 0.0018 |
| | 10 | μM | 78.1 | 5.9 | <0.0001 |
| Day 12 | Control | | 43 | 6.1 | |
| | 0.5 | μM | 45.2 | 7.1 | 0.2494 |
| | 10 | μM | 49.7 | 8.1 | 0.0088 |
| Day 15 | Control | | 18.3 | 5.5 | |
| | 0.5 | μM | 18.3 | 6.2 | 0.2338 |
| | 10 | μM | 23.7 | 5.3 | 0.0048 |

### Example 4: Compound 1 Decreases the Proportion of Inactive C. elegans Through Increasing the Proportion of Moderately Active Animals

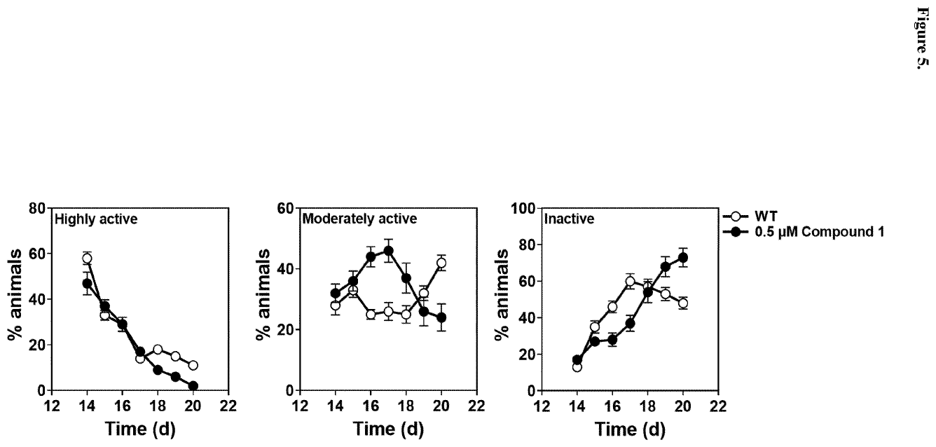

It is possible to determine the proportion of highly active, moderately active and inactive C. elegans at each day of the lifespan and plot these on a graph. FIG. 5, shows that as the ageing population of C. elegans in the control (untreated) population, there is a decrease in highly active worms, and an increase in inactive worms throughout the lifespan. There is a steady-state level of moderately active worms up to middle life of the C. elegans before a small increase.

Interestingly, as shown in FIG. 5, 0.5 μM Compound 1 decreases the proportion of inactive worms, suggesting that Compound 1 can prevent worms from becoming inactive during their lifespan, particularly in mid-life. The ability of 0.5 μM Compound 1 to decrease the number of inactive worms can be mediated through its improvements on moderately active or highly active worms. We see that 0.5 μM Compound 1 increases the number of moderately active worms and not through increases in the proportion of highly active worms. It is important to note that 1) we did not see any change in the proportion of highly active, moderately active and inactive worms before day 14, and we put the cut-off at 20 days as the number of worms living beyond 20 days makes the interpretation of the data difficult and to derive significant differences through under powering.

### Example 5: Compound 1 Decreases the Proportion of Inactive C. elegans Through Increasing the Proportion of Highly Active Animals

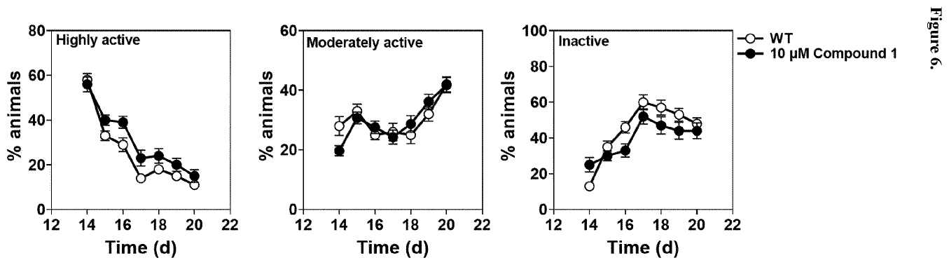

Next we looked at the ability of 10 μM Compound 1 to alter the proportion of highly active, moderately active and inactive worms between days 14 and 20. Interestingly, as shown in FIG. 6, similar to 0.5 μM Compound 1, 10 μM Compound 1 decreases the proportion of inactive worms, again suggesting that Compound 1 can prevent worms from becoming inactive during in mid- to later-life. In contrast to 0.5 μM Compound 1, which mediated this improvement through increasing the proportion of moderately active worms, 10 μM Compound 1 instead increases the proportion of highly active worms. This data shows that 10 μM Compound 1 treated wild-type animals remain highly active beyond the median lifespan. Taken together, these data show that Compound 1 is capable of improving the mobility of C. elegans so that they not only live longer, as shown by the lifespan experiments, but also these worms are more active into mid- and later-life compared to an untreated population. This is particularly interesting because other lifespan extension mechanisms, for example inactivation of insulin signaling through knocking out of the daf-2 or age-1 genes, extend lifespan at the cost of negatively impacting most of the locomotory activity of C. elegans. To this effect, Compound 1 is instead similar to Resveratrol and Urolithin A, which are another examples of lifespan extending compounds that do not negatively impact activity.

### Example 6: Experiments on Autophagy Induction in Zebrafish Larvae

Finally, we monitored the ability of Compound 1 to induce autophagy. Improvements in autophagy have been implicated in the ability of cells and organisms to live longer and maintain better energy homeostasis that may boost improvements in mobility.

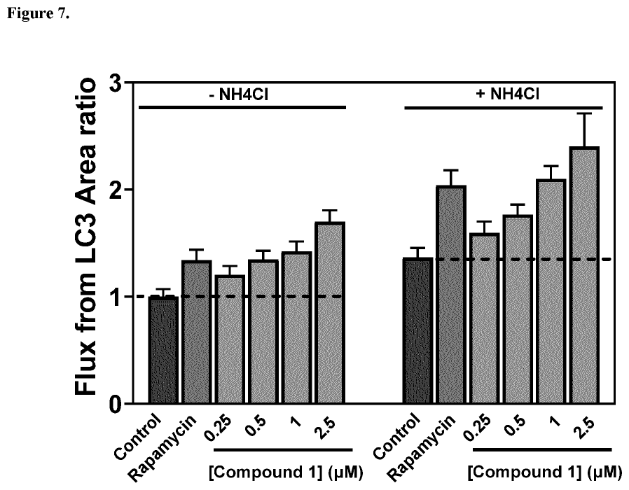

We show FIG. 7 that 1 μM Rapamycin, an inhibitor of mTOR and a well-known autophagy inducer, increased the levels of LC-3 II consistent with Rapamycin's ability to increase autophagy. Interestingly, treatment of Zebrafish larvae with 0.25, 0.5, 1 and 2.5 μM Compound 1, showed a dose-dependent increase in autophagy.

Co-treatment of Zebrafish larvae with NH₄Cl, which can block the degradation of autophagosomes, allows us to capture the ability of compounds to increase autophagy which may have differing rates/kinetics of autophagic flux. This is a standard practice within the autophagy field. As shown in FIG. 7, in the presence of NH₄Cl, 1 μM Rapamycin is capable of increasing LC-3 and autophagy. Similarly, 0.25, 0.5, 1 and 2.5 μM Compound 1, again showed a dose-dependent increase in autophagy in the presence of NH₄Cl. Taken together, this data shows that Compound 1 is a very potent inducer of autophagy in Zebrafish larvae.

1. A method for (i) increasing resistance to age-related pathologies, (ii) improving a physiological state or disorder related to cell ageing, (iii) improving a physiological state linked to metabolic fatigue in one or more cells, (iv) increasing mitochondrial energy in one or more cells, (v) increasing antioxidant capacity, reducing oxidative stress and/or enhancing mitochondrial function, (vi) improving mobility and/or (vii) improving healthspan and/or lifespan in an individual comprising administering a composition comprising a compound having the general formula I, in an effective amount to the individual 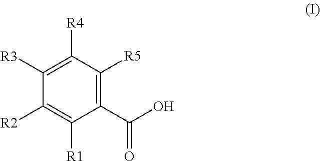
wherein R1 and R3 are each independently selected from the group consisting of OH; OCH3; O-aliphatic saturated or unsaturated acyl, O-glycoside; acylated O-glycoside; sulfated O-glycoside; or a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C2 to C20 alkenyl; substituted and branched, C4 to C20 polyalkenyl; substituted and branched C2 to C20 alkynyl, and substituted and branched C4 to C20 polyalkynyl,
and a derivative or analogue thereof, a OCH3 group can cyclize with a neighboring OH group to form a methylene dioxy bridge.

2. A method for delaying off-set of metabolic decline, maintaining muscle mass and/or muscle function, decreasing oxidative stress, maintaining immune function and/or maintaining cognitive function in a healthy older adult comprising administering a composition comprising at least a compound having the general formula I, in a therapeutically effective amount to the adult, 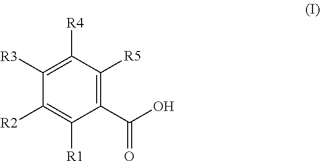
wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C2 to C20 alkenyl; substituted and branched, C4 to C20 polyalkenyl; substituted and branched C2 to C20 alkynyl, or substituted and branched C4 to C20 polyalkynyl.
and a derivative or analogue thereof, a OCH3 group can cyclize with a neighboring OH group to form a methylene dioxy bridge.

3. A method for inducing autophagy comprising administering a compound having the general formula I, 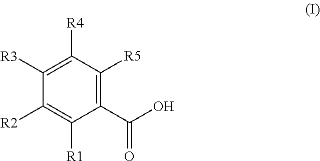
wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C2 to C20 alkenyl; substituted and branched, C4 to C20 polyalkenyl; substituted and branched C2 to C20 alkynyl, substituted and branched C4 to C20 polyalkynyl,
and a derivative or analogue thereof, a OCH3 group can cyclize with a neighboring OH group to form a methylene dioxy bridge.

4. A method according to claim 1, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; substituted and branched C1 to C20 alkyl; substituted and branched, C2 to C20 alkenyl; substituted and branched, C4 to C20 polyalkenyl; substituted and branched C2 to C20 alkynyl, substituted and branched C4 to C20 polyalkynyl,
and a derivative or analogue thereof, a OCH3 group can cyclize with a neighboring OH group to form a methylene dioxy bridge.

5. A method according to claim 1, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2 is selected from the group consisting of H, CH3, OH; O-glycoside; C-glycoside; sulfated O-glycoside; sulfated C-glycoside; a chlorine; a CHO (aldehyde); a sulfate; substituted and branched, C2 to C15 alkenyl; substituted and branched, C4 to C15 polyalkenyl,
R4 is selected from the group consisting of H; CH3, OH; OCH3; a chlorine, or a 3,3-dimethylallyl chain (1h),
R5 is selected from the group consisting of H; substituted and branched C1 to C20 alkyl; substituted and branched, C2 to C20 alkenyl; substituted and branched, C4 to C20 polyalkenyl,
and a derivative or analogue thereof, a OCH3 group can cyclize with a neighboring OH group to form a methylene dioxy bridge.

6. A method according to claim 1, wherein R1 and R3 are each independently selected from the group consisting of OH; OCH3; O-aliphatic saturated or unsaturated acyl, O-glycoside; acylated O-glycoside; sulfated O-glycoside; and a sulfate selected from the group consisting of
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C20 isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
and a derivative or analogue thereof, a OCH3 group can cyclize with a neighboring OH group to form a methylene dioxy bridge.

7. A method according to claim 1, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain selected from the group consisting of: 3,7,11,15-Tetramethylhexadecyl (phytanyl) (4a), (2E,6,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
and a derivative or analogue thereof, a OCH3 group can cyclize with a neighboring OH group to form a methylene dioxy bridge.

8. A method according to claim 1, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2 is selected from the group consisting of CH3, OH; O-glycoside; C-glycoside; sulfated O-glycoside; sulfated C-glycoside; a chlorine; a CHO (aldehyde); a sulfate; a C5 isoprenoid chain among the following representatives: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); and a (substituted) polyalkenyl chain exclusively represented by: 8-(3,4-dihydroxyphenyl)-4,7-octadien-1-yl (18a);
R4 is selected from the group consisting of H; CH3, OH; OCH3; a chlorine, or a 3,3-dimethylallyl chain (1h);
R5 is selected from the group consisting of H; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain among the following representatives: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain among the following representatives: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); and a (substituted) polyalkenyl chain among the following representatives: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a),
and a derivative or analogue thereof, a OCH3 group can cyclize with a neighboring OH group to form a methylene dioxy bridge.

9-10. (canceled)

11. The method according to claim 1, wherein said compound of general formula I is cannabidiolic acid, CAS number 1244-58-2.

12-18. (canceled)

19. A method according to claim 2, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; substituted and branched C1 to C20 alkyl; substituted and branched, C2 to C20 alkenyl; substituted and branched, C4 to C20 polyalkenyl; substituted and branched C2 to C20 alkynyl, substituted and branched C4 to C20 polyalkynyl,
and a derivative or analogue thereof, a OCH3 group can cyclize with a neighboring OH group to form a methylene dioxy bridge.

20. A method according to claim 2, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2 is selected from the group consisting of H, CH3, OH; O-glycoside; C-glycoside; sulfated O-glycoside; sulfated C-glycoside; a chlorine; a CHO (aldehyde); a sulfate; substituted and branched, C2 to C15 alkenyl; substituted and branched, C4 to C15 polyalkenyl,
R4 is selected from the group consisting of H; CH3, OH; OCH3; a chlorine, or a 3,3-dimethylallyl chain (1h),
R5 is selected from the group consisting of H; substituted and branched C1 to C20 alkyl; substituted and branched, C2 to C20 alkenyl; substituted and branched, C4 to C20 polyalkenyl,
and a derivative or analogue thereof, a OCH3 group can cyclize with a neighboring OH group to form a methylene dioxy bridge.

21. A method according to claim 2, wherein R1 and R3 are each independently selected from the group consisting of OH; OCH3; O-aliphatic saturated or unsaturated acyl, O-glycoside; acylated O-glycoside; sulfated O-glycoside; and a sulfate selected from the group consisting of
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C20 isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
and a derivative or analogue thereof, a OCH3 group can cyclize with a neighboring OH group to form a methylene dioxy bridge.

22. A method according to claim 2, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
and a derivative or analogue thereof, a OCH3 group can cyclize with a neighboring OH group to form a methylene dioxy bridge.

23. A method according to claim 2, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2 is selected from the group consisting of CH3, OH; O-glycoside; C-glycoside; sulfated O-glycoside; sulfated C-glycoside; a chlorine; a CHO (aldehyde); a sulfate; a C5 isoprenoid chain among the following representatives: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); and a (substituted) polyalkenyl chain exclusively represented by: 8-(3,4-dihydroxyphenyl)-4,7-octadien-1-yl (18a);
R4 is selected from the group consisting of H; CH3, OH; OCH3; a chlorine, or a 3,3-dimethylallyl chain (1h);
R5 is selected from the group consisting of H; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain among the following representatives: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain among the following representatives: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); and a (substituted) polyalkenyl chain among the following representatives: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a),
and a derivative or analogue thereof, a OCH3 group can cyclize with a neighboring OH group to form a methylene dioxy bridge.

24. The method according to claim 2, wherein said compound of general formula I is cannabidiolic acid, CAS number 1244-58-2.

25. A method according to claim 3, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; substituted and branched C1 to C20 alkyl; substituted and branched, C2 to C20 alkenyl; substituted and branched, C4 to C20 polyalkenyl; substituted and branched C2 to C20 alkynyl, substituted and branched C4 to C20 polyalkynyl,
and a derivative or analogue thereof, a OCH3 group can cyclize with a neighboring OH group to form a methylene dioxy bridge.

26. A method according to claim 3, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2 is selected from the group consisting of H, CH3, OH; O-glycoside; C-glycoside; sulfated O-glycoside; sulfated C-glycoside; a chlorine; a CHO (aldehyde); a sulfate; substituted and branched, C2 to C15 alkenyl; substituted and branched, C4 to C15 polyalkenyl,
R4 is selected from the group consisting of H; CH3, OH; OCH3; a chlorine, or a 3,3-dimethylallyl chain (1h),
R5 is selected from the group consisting of H; substituted and branched C1 to C20 alkyl; substituted and branched, C2 to C20 alkenyl; substituted and branched, C4 to C20 polyalkenyl,
and a derivative or analogue thereof, a OCH3 group can cyclize with a neighboring OH group to form a methylene dioxy bridge.

27. A method according to claim 3, wherein R1 and R3 are each independently selected from the group consisting of OH; OCH3; O-aliphatic saturated or unsaturated acyl, O-glycoside; acylated O-glycoside; sulfated O-glycoside; and a sulfate selected from the group consisting of
R2, R4, and R5 are each independently H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C20 isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
and a derivative or analogue thereof, a OCH3 group can cyclize with a neighboring OH group to form a methylene dioxy bridge.

28. A method according to claim 3, wherein R1 and R3 are each independently selected from the group consisting of OH; OCH3; O-aliphatic saturated or unsaturated acyl, O-glycoside; acylated O-glycoside; sulfated O-glycoside; and a sulfate,
R2, R4, and R5 are each independently selected from the group consisting of H, OH; OCH3; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C5 isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C15 isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C20 isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
and a derivative or analogue thereof, a OCH3 group can cyclize with a neighboring OH group to form a methylene dioxy bridge.

29. The method according to claim 3, wherein said compound of general formula I is cannabidiolic acid, CAS number 1244-58-2.
